(19) United States
(12) Patent Application Publication
(10) Pub. No.: US 2025/0332294 A1
(43) Pub. Date: Oct. 30, 2025

(54) Cancer-Targeted Alpha-Particle Therapeutic Vehicles For Treatment Of Cancer

(71) Applicant: Board of Trustees of Michigan State University, East Lansing, MI (US)

(72) Inventors: Kurt R. ZINN, East Lansing, MI (US); Nate kauffman, Grand Rapids, MI (US); Satyendra Singh, east Lansing, MI (US); jinda Fan, Okemos, MI (US)

(21) Appl. No.: 19/259,709

(22) Filed: Jul. 3, 2025

Related U.S. Application Data
(63) Continuation of application No. PCT/US2024/023819, filed on Apr. 10, 2024.
(60) Provisional application No. 63/458,241, filed on Apr. 10, 2023.

(51) Int. Cl. A61K 51/04; A61K 38/38; A61K 39/395; A61P 35/00

(52) U.S. Cl. CPC A61K 51/0482; A61K 38/38; A61K 39/39558; A61P 35/00; A61K 2121/00

(57) ABSTRACT

Disclosed herein are conjugates comprising an alpha particle emitter and an anti-cancer agent, compositions comprising the conjugate, kits for radiolabeling with an alpha particle emitter and an anti-cancer agent, and a method of using the conjugate to treat cancer. The alpha-particle emitter and the anti-cancer agent may be unmodified or linked to each other with a chelating agent.

### CROSS-REFERENCE TO RELATED APPLICATIONS

This application is a continuation of PCT International Application No. PCT/US2024/023819, filed Apr. 10, 2024, which claims the benefit of U.S. Provisional Patent Application No. 63/458,241, filed Apr. 10, 2023. The patent applications identified in this paragraph are incorporated herein by reference in their entirety.

### FIELD

The field of the invention relates to a conjugate comprising an alpha-particle emitter and an anti-cancer agent. The conjugate may be used to treat cancer.

### BACKGROUND

The background description includes information that may be useful in understanding the compositions and methods described herein. It is not an admission that any of the information provided herein is prior art or relevant to the compositions and methods, or that any publication specifically or implicitly referenced is prior art.

Radiation therapy is a pillar of oncological care. External beam radiation therapy is a well-established modality for tumor therapy and efficiency. Even with these advancements, external beam has limitations such as treating tumors near sensitive or mobile organs, deep seated tumors, or wide-spread metastases. Efforts to deliver radiation treatment in these situations have led to alternative radiotherapy technologies. Brachytherapy and Selective Internal Radiation Therapy (SIRT) place radioactive sources or drugs near or within tumors. An emerging alternate approach is targeted intravenous delivery of small peptides radiolabeled with beta- (β-) emitters to address situations where external beam is contraindicated. One of the oldest and most common therapies for thyroid cancer is ¹²³I-iodide for imaging and dosimetry followed by ¹³¹I-iodide for radiation therapy. This concept of imaging first, then selecting appropriate therapy has evolved into “radiotheranostics” and is rapidly advancing by using novel targeting strategies and new therapeutic radioisotopes.

Alpha (α)-particles, which are comprised of 2 neutrons and 2 protons, are emitted from large unstable radioisotopes during decay. α-emitting radionuclides (α-particle emitters) have not been used widely clinically because of the lack of commercial availability and lack of pure α-emitting nuclides. α-particles are attractive from a cancer biology standpoint because of three major benefits compared to those that decay by only β-emissions: high linear energy transfer (LET), short penetration range and efficiency in hypoxic environments. The higher LET means more of the total radiation dose is delivered over an equal pathlength. α-particles can deliver up to 1000× more dose to cells than β-particles with the same number of radioactive decays. This high strength allows for double rather than single strand DNA breaks, leading to increased cell death. Cancer cells can adapt to single stranded DNA breaks and survive, but struggle when double strand breaks occur.

The short path length of α-particles is useful for therapy. α-particles deliver their energy over microns of tissue penetration while β-can penetrate millimeters deep. Sensitive tissues near solid tumor locations, including prostate cancers, can be heavily irradiated during β-therapy. Use of radioisotopes with α-particles may reduce the off-targeting effect while still maintaining therapeutic efficacy in the targeted tumors.

### SUMMARY

A conjugate comprising an alpha-particle emitter and an anti-cancer agent is disclosed herein. The alpha-particle emitter may be a ²⁰⁹Bi, ²¹²Bi, ²¹³Bi, ²¹⁴Bi, ²¹¹Pb, ²¹²Pb, ²¹⁴Pb, ²¹⁴Po, ²¹¹At, ²²⁵Ac, ²²⁷Th, ²²²Rn, ²²³Ra, or ²²⁴Ra, or a combination of two or more emitters, such as ²¹²Pb/²¹⁴Pb, ²¹⁴Bi/²¹⁴Pb, ²¹²Bi/²¹²Pb, ²²⁴Ra/²¹²Bi, ²²⁷Th/²²³Ra, or ²²⁵Ac/²¹³Bi. The anti-cancer agent may be a HER2 inhibitor, including an anti-HER2 antibody such as trastuzumab or biosimilars or bioequivalents thereof, an EGFR inhibitor, including an anti-EGFR antibody such as cetuximab, panitumumab, zalutumumab, nimotuzumab, and matuzumab, or macroagglutinated albumin. The conjugate may further comprise a chelator, such as TCMC, DOTA, or DTPA.

A pharmaceutical composition comprising the conjugate is also described herein. The pharmaceutical composition may comprise the conjugate along with one or more pharmaceutically acceptable excipients. The excipients may be a pH buffer, stabilizer, antioxidant, diluent, carrier, detergent, surfactant, or combinations thereof. The pharmaceutical composition may be formulated for administration, such as for intravesical administration.

Various objects, features, aspects, and advantages will become more apparent from the following detailed description of preferred embodiments, along with the accompanying drawings.

### BRIEF DESCRIPTION OF THE DRAWINGS

FIG. 1 is a representative in vivo image of a mouse injected with luciferase-positive SKOV-3 cancer cells.

FIG. 2 depicts the normalized tumor signal of mice receiving a single dose of ²¹⁴Pb/²¹⁴Bi-TCMC-trastuzumab, ²¹⁴Pb/²¹⁴Bi-TCMC-IgG isotype antibody, or control (i.e., no treatment).

FIG. 3 depicts the normalized tumor signal of mice receiving two doses of ²¹⁴Pb/²¹⁴Bi-TCMC-trastuzumab, ²¹⁴Pb/²¹⁴Bi-TCMC-IgG isotype antibody, or control (i.e., no treatment).

FIG. 4 depicts the tumor signal of individual mice following inoculation with SKOV-3 cells followed by two doses of ²¹⁴Pb/²¹⁴Bi-TCMC-trastuzumab.

FIG. 5A depicts the accumulation of ²¹²Bi-MAA in various tissues of mice inoculated with 4T1 cells at 2 and 4 hours after administration. FIG. 5B depicts the accumulation of ²¹²Bi-MAA in various tissues of mice inoculated with EO771 cells at 2 and 4 hours after administration. FIG. 5C depicts the total radiation dose percentage in tumors of mice inoculated with 4T1 or EO771 cells.

FIG. 6A depicts the normalized tumor size of EO771 tumors in mice treated with ²¹²Bi-MAA. FIG. 6B depicts the normalized tumor size of 4T1 tumors in mice treated with ²¹²Bi-MAA.

FIG. 7 depicts the clonogenic assay of human bladder cells untreated or treated with ²¹⁴Pb/²¹⁴Bi-TCMC-trastuzumab/cetuximab.

FIG. 8A depicts the crystal violet staining of treated cells showing total number of colonies in untreated cells or cells treated with 10 μCi or 40 μCi ²¹⁴Pb-MAA. FIG. 8B depicts the total radiance from each treated sample (n=3, error bars reported as standard error). FIG. 8C depicts total number of colonies after manual counting of stained cells (n=3, error bars reported as standard error).

FIG. 9A-E show the saturation binding curves for the binding of ²⁰³Pb-TCMC-trastuzumab to the HER2+ SKOV3 cells (human ovarian cancer cell line) after radiolabeling at different temperatures and durations.

### DETAILED DESCRIPTION

Definitions: The following definitions refer to the various terms used above and throughout the disclosure. As used herein, all nouns in singular form are intended to convey the plural and all nouns in plural form are intended to convey the singular, except where context clearly indicates otherwise. As used herein, “and/or” includes any and all combinations of one or more of the associated listed items.

As used herein, “effective amount” refers to the amount, dosage, and/or dosage regime of the conjugate in a composition that is sufficient to induce a desired clinical and/or therapeutic outcome, for example to treat, inhibit, slow the growth of, or reduce the size of cancer. The effective amount may also refer to the amount, dosage, and/or dosage regime of the alpha-particle emitter, the anti-cancer agent, or both. Where the effective amount is based upon the alpha-particle emitter, the effective amount may be calculated by the emitted radiation. The radiation may be measured in by the amount of radiation emitted, e.g., curie (Ci) or becquerel (Bq). The radiation may also be measured by the amount of radiation absorbed, e.g., rad or gray (Gy). Where the effective amount is based on the anti-cancer agent, the effective amount may be based on the amount (e.g., mg or mg/kg) or concentration (e.g., mg/mL) of the anti-cancer agent administered.

As used herein, “alpha-particle emitter” refers to a radioactive agent that emits alpha-particles (α-particles). The alpha-particle emitter may emit short-lived alpha-particles and is not particularly limited and may be any one of ²⁰⁹Bi, ²¹²Bi, ²¹³Bi, ²¹⁴Bi, ²¹¹Pb, ²¹²Pb, ²¹⁴Pb, ²¹⁴Po, ²¹¹At, ²²⁵Ac, ²²⁷Th ²²²Rn, ²²³Ra, or ²²⁴Ra. Alternatively or additionally, the alpha-particle emitter may be a combination of two or more radioactive elements. For example, the alpha-particle emitter may be one of ²¹⁴Pb/²¹²Pb, ²¹⁴Pb/²¹⁴Bi, ²¹²Pb/²¹²Bi, ²²⁴Ra/²¹²Bi, ²²⁷Th/²²³Ra, or ²²⁵Ac/²¹³Bi. The alpha-particle emitter may be from a generator system, such as a ²²⁴Ra generator, ²²⁵Ac generator, and ²²²Rn generator.

As used herein, “anti-cancer agent” refers to an active pharmaceutical ingredient that shows a capacity to treat, inhibit, slow the growth of, or reduce the size of cancer. The anti-cancer agent may be an antibody, such as a monoclonal antibody (mAb) or a polyclonal antibody (pAb), that binds to or otherwise targets a cancer cell and/or antigen of a cancer cell. The anti-cancer agent may be an antibody that binds to HER2 and/or epidermal growth factor receptor (EGFR). Anti-HER2 antibodies include, but are not limited to, trastuzumab (HERCEPTIN®), trastuzumab-anns (KANJINTI®), trastuzumab-dkst (OGIVRI®), trastuzumab-qyyp (TRAZIMERA®), or trastuzumab-pkrb (HERZUMA®). Anti-HER2 antibodies may also include conjugates, including trastuzumab emtansine (KADCYLA®) and trastuzumab deruxtecan (ENHERTU®). Anti-EGFR antibodies include, but are not limited to, cetuximab (ERBITUX®), biosimilars of cetuximab such as ABP 494 (from Actavis/Amgen), CT-P15 (from Celtrion), and STI-001 (from MabTech), panitumumab (VECTIBI®), zalutumumab, nimotuzumab, or matuzumab. Alternatively, the anti-cancer agent may be macroaggregated albumin (MAA).

As used herein, “chelator” refers to a compound that coordinates or otherwise interacts with a metal, such as a radioactive metal. The chelator is not particularly limited and includes 2-[4,7,10-tris(2-amino-2-oxoethyl)-1,4,7,10-tetrazacyclododec-1-yl]acetamide (TCMC, also known as DOTAM), 2,2′,2″,2′″-(1,4,7,10-tetraazacyclododecane-1,4,7,10-tetrayl)tetraacetic acid (DOTA), and 2,2′,2″,2′″-{[(Carboxymethyl)azanediyl]bis(ethane-2,1-diylnitrilo)}tetraacetic acid (diethylenetriamine pentaacetate or DTPA).

As used herein, “kit” refers to an assembly of materials that are used in preparing a final drug product for cancer treatment. The materials may be an anti-cancer agent, an alpha-particle emitter, a pH buffer, a chelator, and a stabilizer. The reagents can be provided in a packaged combination in the same or in separate containers, depending on their cross-reactivities and stabilities, and in liquid or in lyophilized form, as appropriate. The amounts and proportions of reagents provided in the kit can be selected so as to provide optimum results for a particular application. The containers may be shielded, such as for transportation and/or storage, to prevent exposure to radiation emitted from the alpha-particle emitter. The kit may further comprise calibration, control materials, and instructions for use.

As used herein, “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In certain embodiments, the subject can be human (e.g., adult male, adult female, adolescent male, adolescent female, male child, female child) under the care of a physician or other health worker. In certain embodiments the subject may not be under the care of a physician or other health worker. The subject may have undergone surgery, received orthopedic treatment, received ophthalmic treatment, or suffering from injury or chronic disease. Alternatively, where the subject is a laboratory mammal, the conjugate may be provided to the laboratory mammal to achieve a scientific understanding rather than a clinical benefit.

Conjugates: The conjugates described herein comprise an alpha-particle emitter and an anti-cancer agent. The alpha-particle emitter and anti-cancer agent may be present in an alpha-particle emitter:anti-cancer agent ratio of about 5:1, about 4:1, about 3:1, about 2:1, about 1:1, about 1:2, about 1:3, about 1:4, and about 1:5. The conjugate may further comprise a chelator.

The alpha-particle emitter may be one or more of ²⁰⁹Bi, ²¹²Bi, ²¹³Bi, ²¹⁴Bi, ²¹¹Pb, ²¹²Pb, ²¹⁴Pb, ²¹⁴Po, ²¹¹At, ²²⁵Ac, ²²⁷Th, ²²²Rn, ²²³Ra, or ²²⁴Ra. Additionally or alternatively, the alpha-particle emitter may be a combination of two or more alpha-particle emitters, such as ²¹⁴Pb/²¹²Pb, ²¹⁴Pb/²¹⁴Bi, ²¹²Pb/²¹²Bi, ²²⁴Ra/²¹²Bi, ²²⁷Th/²²³Ra, or ²²⁵Ac/²¹³Bi. In a preferred embodiment the alpha-particle emitter may be ²¹²Bi, ²¹³Bi, ²¹⁴Bi, ²¹²Pb, ²¹⁴Pb, ²¹⁴Pb/²¹²Pb, ²¹⁴Pb/²¹⁴Bi, or ²¹²Pb/²¹²Bi. In a particular embodiment, the alpha-particle emitter is ²¹⁴Pb. Alternatively, the alpha-particle emitter may be from a ²²⁴Ra generator system.

The anti-cancer agent may be a compound, protein, nucleotide, or a combination thereof. In an embodiment, the anti-cancer agent may be a HER2 inhibitor (e.g., an anti-HER2 antibody), an EGFR inhibitor (e.g., an anti-EGFR antibody), or macroaggregated albumin (MAA). The anti-HER2 or anti-EGFR antibody may be a mAb or a pAb. The anti-HER2 antibody may be, for example, trastuzumab, or a derivative of trastuzumab, such as trastuzumab-anns (KANJINTI®), trastuzumab-dkst (OGIVRI®), trastuzumab-qyyp (TRAZIMERA®), or trastuzumab-pkrb (HERZUMA®). The anti-HER2 antibody may be an antibody conjugated to another active agent, such as trastuzumab emtansine (KADCYLA®) and trastuzumab deruxtecan (ENHERTU®). The anti-EGFR antibody may be, for example, cetuximab (ERBITUX®), panitumumab (VECTIBI®), zalutumumab, nimotuzumab, or matuzumab. In a particular embodiment the HER2 inhibitor is trastuzumab and the alpha-particle emitter may be ²¹²Bi, ²¹³Bi, ²¹⁴Bi, ²¹²Pb, ²¹⁴Pb, ²¹²Pb/²¹⁴Pb, ²¹⁴Bi/²¹⁴Pb, or ²¹²Bi/²¹²Pb. In another embodiment, the anti-cancer agent may be MAA.

The conjugate described herein may further comprise a chelator. The chelator is not particularly limited and may be 2-[4,7,10-tris(2-amino-2-oxoethyl)-1,4,7,10-tetrazacyclododec-1-yl]acetamide (TCMC) 2,2′,2″,2′″-(1,4,7,10-Tetraazacyclododecane-1,4,7,10-tetrayl)tetraacetic acid (DOTA), or 2,2′,2″,2′″-{[(Carboxymethyl)azanediyl]bis(ethane-2,1-diylnitrilo)}tetraacetic acid (diethylenetriamine pentaacetate or DTPA.

The combination of alpha-particle emitter and anti-cancer agent and chelator, if present, is not particularly limited. For example, any one of the preferred alpha-particle emitters (e.g., ²¹²Bi, ²¹³Bi, ²¹⁴Bi, ²¹²Pb ²¹⁴Pb, ²¹⁴Pb/²¹²Pb, ²¹⁴Pb/²¹⁴Bi, or ²¹²Pb/²¹²Bi) may be combined with any one of the preferred anti-cancer agents (e.g., trastuzumab, cetuximab, or MAA). In an embodiment, conjugate may be ²¹⁴Pb/²¹⁴Bi-TCMC-trastuzumab. In another embodiment, the conjugate may be ²¹⁴Pb/²¹⁴Bi-cetuximab/trastuzumab. In a still further embodiment, the conjugate may be ²¹⁴Pb/²¹⁴Bi-MAA.

Compositions: The conjugates described herein may be formulated in a pharmaceutical composition with one or more pharmaceutically acceptable excipients. The pharmaceutical excipient may be a pH buffer, stabilizer, antioxidant, diluent, carrier, detergent, surfactant, or combinations thereof. In particular, the pharmaceutically acceptable excipient may be a pH buffer and a stabilizer. In a specific embodiment, the anti-cancer agent of the conjugate may be trastuzumab and the alpha-particle emitter of the conjugate may be ²¹²Bi, ²¹³Bi, ²¹⁴Bi, ²¹²Pb, ²¹⁴Pb, ²¹⁴Pb/²¹²Pb, ²¹⁴Pb/²¹⁴Bi, or ²¹²Pb/²¹²Bi. In a preferred embodiment, the conjugate of the composition comprises trastuzumab, ²¹⁴Pb, and TCMC or DOTA.

Kits: The conjugate described herein may be provided in a kit, wherein the alpha-particle emitter and the anti-cancer agent are provided in separate containers (e.g., a two-vial kit). The vials of the two-vial kit may further comprise shielding material to block or inhibit the radiation from the alpha-particle emitter. Each container in the two-vial kit may further independently comprise one or more of a buffer for pH adjustment and radiolysis protection, bulking agent, cryoprotectant/lyoprotectant, and/or surfactant to protect antibody from aggregation and denaturation.

Alternatively, the kit may comprise a single container (e.g., a one vial kit). In some embodiments, the one vial kit may comprise an alpha-particle emitter and an antibody. The one vial kit may further comprise one or more of a buffer for pH adjustment and radiolysis protection, bulking agent, cryoprotectant/lyoprotectant, and/or surfactant to protect antibody from aggregation and denaturation.

The kit (e.g., two vial kit or one vial kit) may further comprise written material (e.g., instructions). The kit may be configured such that the containers comprising the alpha-particle emitter and the anti-cancer agent are configured to combine the alpha-particle emitter and the anti-cancer agent to radiolabel the anti-cancer agent with the alpha-particle emitter. The kit may further comprise a device, which may be the container itself, configured to provide the radiolabeled anti-cancer agent to a patient. The contents of the kit may be freeze dried.

Methods: The conjugate described herein may be provided to a patient suffering from cancer to treat, alleviate, inhibit, and/or reduce the growth of the cancer. The patient may suffer from bladder cancer, ovarian cancer, breast cancer, skin cancer, prostate cancer, pancreatic cancer, bone cancer, stomach cancer, lung cancer, and/or brain cancer. The conjugate may be provided at a dose of about 10 to about 20,000 μCi.

### EXAMPLES

The following examples are provided to further illustrate the fusion peptide disclosed herein but should not be construed as in any way limiting its scope.

### Example 1: Treatment of Ovarian Cancer with Single Dose of ²¹⁴Pb/²¹⁴Bi-TCMC-Trastuzumab Conjugate

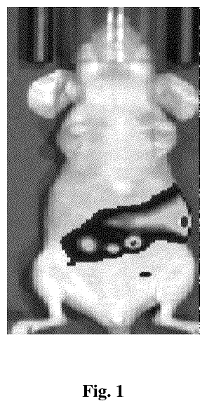

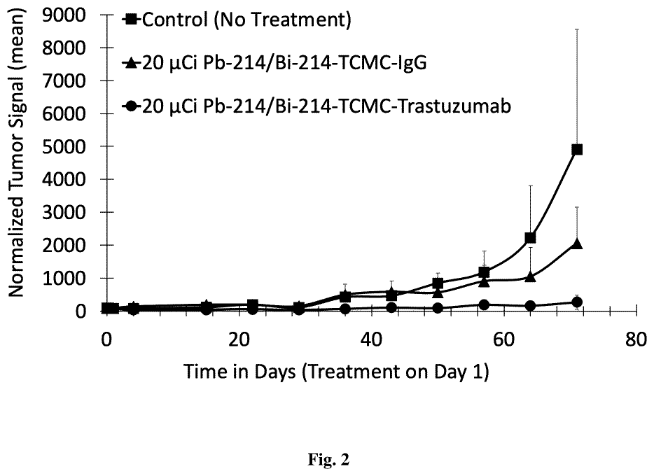

Female nude mice (8 wks from Charles River Laboratories) were implanted with 5×10⁶ luciferase-positive SKOV-3 cells via intraperitoneal injection. After 5 weeks, the mice were separated and randomly assigned to one of the following treatment groups based on bioluminescence: I: 20 μCi (0.74 MBq)²¹⁴Pb/²¹⁴Bi-TCMC-trastuzumab; II: 20 μCi (0.74 MBq) ²¹⁴Pb/²¹⁴Bi-TCMC-IgG isotype antibody; and III: untreated (5-6 mice/group). The mice in Groups I and II were administered 1 dose of treatment (IP) three weeks (Day 27) after implantation of the SKOV-3 cells. The mice were imaged weekly (PE Spectrum) and tumor signal was measured by region of interest analyses. Each mouse had tumor signals after treatment normalized to tumor signal prior to starting treatment. A representative in vivo image of the tumor is shown in FIG. 1.

As shown in FIG. 2, mice treated with ²¹⁴Pb/²¹⁴Bi-TCMC-trastuzumab had significant long-term reduction in tumor growth as compared to control mice or mice receiving the ²¹⁴Pb/²¹⁴Bi-TCMC-IgG isotype antibody. These data shows that the benefit of treatment relies on the anti-cancer agent targeting the tumor (e.g., trastuzumab targeting HER2).

### Example 2: Treatment of Ovarian Cancer with Double Dose of ²¹⁴Pb/²¹⁴Bi-TCMC-Trastuzumab Conjugate

Female nude mice (8 wks from Charles River Laboratories) were implanted with 5×10⁶ luciferase-positive SKOV-3 cells via intraperitoneal injection. After 5 weeks, the mice were separated and randomly assigned to one of the following treatment groups based on bioluminescence: I: 20 μCi (0.74 MBq)²¹⁴Pb/²¹⁴Bi-TCMC-trastuzumab; II: 20 μCi (0.74 MBq) ²¹⁴Pb/²¹⁴Bi-TCMC-IgG isotype antibody; and III: untreated (7 mice/group). The mice in Groups I and II were administered 2 dose of treatment (IP) at days 35 and 43 after implantation of the SKOV-3 cells. The mice were imaged weekly (PE Spectrum) and tumor signal was measured by region of interest analyses. Each mouse had tumor signals after treatment normalized to tumor signal prior to starting treatment.

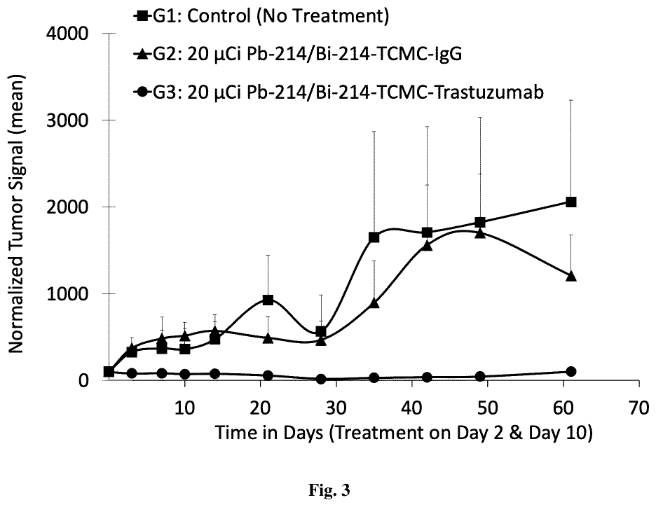

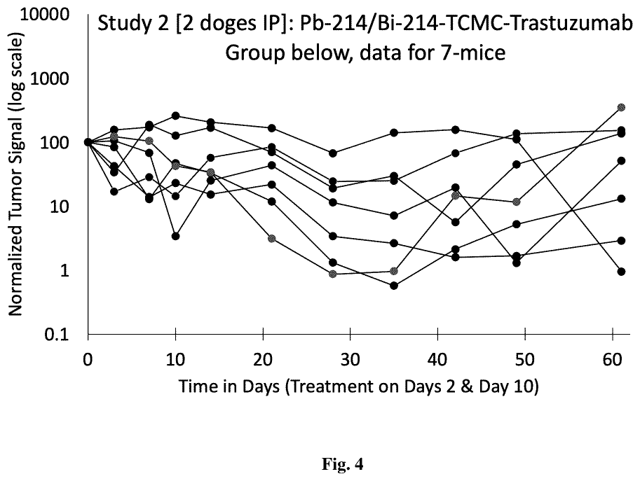

As shown in FIG. 3, mice treated with ²¹⁴Pb/²¹⁴Bi-TCMC-trastuzumab had significant long-term reduction in tumor growth as compared to control mice or mice receiving the ²¹⁴Pb/²¹⁴Bi-TCMC-IgG isotype antibody. Two doses of ²¹⁴Pb/²¹⁴Bi-TCMC-trastuzumab showed a more than 50-fold reduction in tumor signal at day 34 following the first dose of treatment. FIG. 4 shows the tumor signal for each mouse in the ²¹⁴Pb/²¹⁴Bi-TCMC-trastuzumab treatment group on a log scale. These data shows that the benefit of treatment relies on the anti-cancer agent targeting the tumor (e.g., trastuzumab targeting HER2).

### Example 3: Treatment of Cancer with ²¹²Bi-MAA

FDA-approved MAA kits (Pulmontech) were purchased from Cardinal Health (East Lansing, MI). The ²²⁴Ra/²¹²Pb generators (5 mCi) were provided by Oak Ridge. The generator was washed with 500 μL of 2 M HCl upon receival. Every day afterwards, ²¹²Bi was eluted from the generator with 800 μL of 0.15M KI/0.1 M HCl solution. The eluent was treated with 8 M HNO₃ and evaporated to dryness 3 times. The dried vials containing the ²¹²Bi were reconstituted with 100 μL of 0.1 M HNO₃ for transfer to vials containing 10 μL of 1 M NaOH for neutralization. ²¹²Pb from the generator was evaluated with a gamma counter (Wizard2, Perkin Elmer) using a window around the gamma-ray energy of ²¹²Pb which differed from ²¹²Bi. ²¹²Bi half-life was also confirmed by repeatedly measuring a ²¹²Bi sample over time with a dose calibrator (CRC-25R, Capintec).

For radiolabeling MAA with ²¹²Bi, 3 mg of the MAA kit (0.33 mg aggregated albumin) was resuspended in 500 μL 1×PBS and added to the neutralized ²¹²Bi. The ²¹²Bi-MAA solution was incubated for 10 minutes at 70° C. with 500 RPM shaking. ²¹²Bi-bound MAA was purified by centrifugation at 1000 g for 5 minutes with the pellet containing the ²¹²Bi bound MAA and the supernatant containing unbound ²¹²Bi that was easily removed. The percentage of ²¹²Bi bound to MAA was determined with iTLC using 10 mM EDTA in 0.15 M NH₄OAc as the mobile phase.

Balb/c and C57BL/6 mice (8 weeks, from Charles River Laboratory) were implanted with 1×10⁵ 4T1 and EO771 Luc+ cells, respectively, in the fourth mammary gland. After 7 and 8 days post implantation, the 4T1 and EO771 tumors, respectively, were intratumorally injected with 50 or 100 μCi of ²¹²Bi-MAA and vehicle control (MAA alone) suspended in 20 μL of 0.9% saline using 25 gauge integrated needle syringes with zero dead volume. All groups were euthanized once the tumor size reached 2 cm in length in any group. EO771 mice were injected intraperitoneally with 1.5 mg of luciferin 10 minutes prior to sacrifice to allow for ex vivo BLI on IVIS Spectrum. Imaging was done using auto exposure and data was analyzed using ROI and radiance.

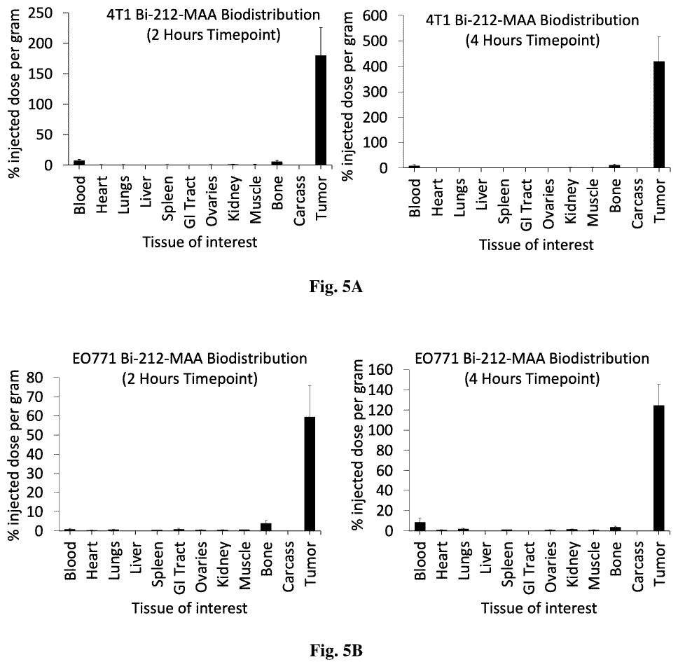

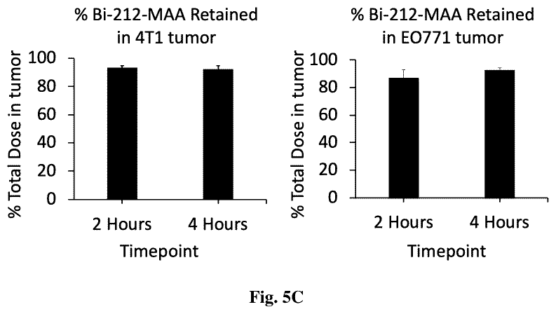

FIG. 5A shows the biodistribution of ²¹²Bi-MAA biodistribution in mice inoculated with 4T1 cells at 2 and 4 hours following administration of the alpha-particle emitter. FIG. 5B shows the biodistribution of ²¹²Bi-MAA biodistribution in mice inoculated with EO771 cells at 2 and 4 hours following administration of the alpha-particle emitter. These results show that the ²¹²Bi-MAA concentrates in the tumor with virtually no accumulation in other tissues. FIG. 5C shows the total amount of recovered activity present in the tumors at 2 and 4 hours post injection.

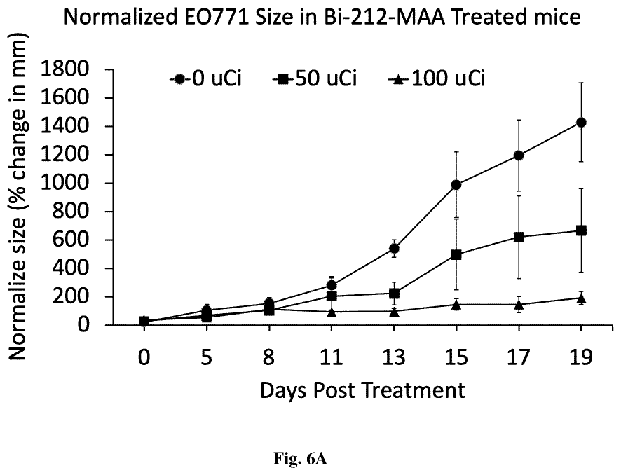

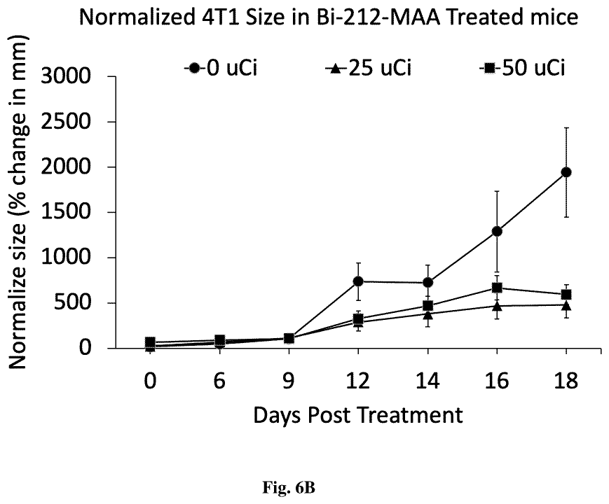

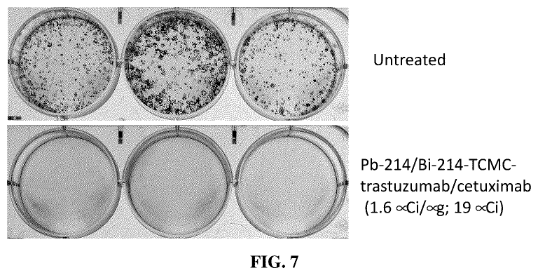

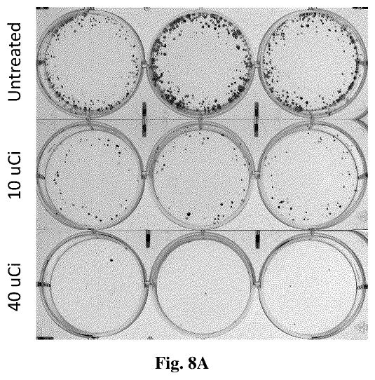

FIG. 6A shows the tumor growth in mice inoculated with EO771 cells following administration of 0 μCi (control), 50 μCi, or 100 μCi of ²¹²Bi-MAA. FIG. 6B shows the tumor growth in mice inoculated with 4T1 cells following administration of 0 μCi (control), 25 μCi, or 50 μCi of ²¹²Bi-MAA. These results show that administration of ²¹²Bi-MAA inhibited tumor growth in mice in a dose-dependent manner. Mouse weight, an index for toxicity of the ²¹²Bi-MAA, increased in all treatment groups, indicating that there was no systemic toxicity of the treatment.

### Example 4: Treatment of Human Ovarian Cancer with Low HER2 Expression with ²¹⁴Pb/²¹⁴Bi-Labeled Trastuzumab

Saturation binding assays were performed following ^{99m}Tc labeling of Trastuzumab (HYNIC method) to determine binding affinity (Kd) and total receptors/cell; 1:2 serial dilution series included three unblocked replicates/dilution and one blocked replicate/dilution (>100 molar fold). Total cells per well were determined using ATPlite luminescence assays. Trastuzumab antibody and isotype-matched control antibody (IgG) were conjugated with 2-(4-isothiocyanotobenzyl)-1,4,7,10-tetraaza-1,4,7,10-tetra-(2-carbamoylmethyl)-cyclododecane (TCMC: Ab; 6:1 molar ratio), radiolabeled with ²¹⁴Pb/²¹⁴Bi, then purified with zeba desalting column 40K MWCO (2 mL). ²¹⁴Pb/²¹⁴Bi bound to Ab (purity) was measured by iTLC. Binding assays for ²¹⁴Pb/²¹⁴Bi-TCMC-trastuzumab used 96-well break-apart plates coated with ErbB2/Fc Chimera (n=7). Female nude mice (8 wks) implanted intraperitoneally (IP) with ˜4 million luciferase+OVCAR-3 cells 9 weeks earlier were randomly assigned to 3 equal groups based on bioluminescence signal (n=6-7/group), injected 2× IP (Day 1 and Day 9) with nothing (G1-untreated control), 20 μCi ²¹⁴Pb/²¹⁴Bi-TCMC-IgG (G2), 20 μCi ²¹⁴Pb/²¹⁴Bi-TCMC-Trastuzumab (G3). Cells and mice were imaged over time with an IVIS Spectrum; the signal for each mouse was normalized to its signal before starting treatment, thus each mouse started at 100%.

Binding assays confirmed specific and high-affinity binding of ^{99m}Tc-Trastuzumab to OVCAR3 cells (Kd=3.3±0.7 nM) with 7900±770 receptors/cell. Trastuzumab and isotype-matched IgG were radiolabeled with ²¹⁴Pb/²¹⁴Bi and purified in 25 minutes, in high yield and purity (>97%), Specific activity ranged from 2-3 μCi/g. ²¹⁴Pb/²¹⁴Bi-TCMC-Trastuzumab retained high-affinity, specific binding to ErbB2. Mice at 12 wks after treatment showed the mean tumor signal for G3 decreased to 57% of starting signal. The mean tumor signal in G2 decreased to 77% and G1 increased to 254%, compared with starting signal. There was no toxicity as determined by weight loss with all animals surviving to 72 days. After 12 weeks, the G2 and G3 showed significant and similar treatment efficacy for the 72-day observation period.

### Example 5: Evaluation of ²¹⁴Pb/²¹⁴Bi-TCMC-Cetuximab/Trastuzumab for Killing Human Bladder Cancer Cells

Binding assays are performed using ^{99m}Tc-labeled cetuximab or ⁹⁹mTc-labeled trastuzumab to establish EGFR1 and EGFR2 levels on a panel of human bladder cancer cell lines. With information obtained from the binding assay, clonogenic assays are conducted with two human bladder cancer cells lines and one control cell line, either a bladder epithelial cell line or fibroblast cell line negative for EGFR1 and EFR2. This assay has been successfully developed for human bladder cancer cells lines. As shown below in FIG. 7, about 1000 SCaBER human bladder cancer cells were seeded a few hours before addition of ²¹⁴Pb/²¹⁴Bi-TCMC-cetuximab/trastuzumab. Colony formation was evaluated after 10 days. FIG. 7 shows the treatment prevented colony formation. ²¹⁴Pb/²¹⁴Bi-TCMC-cetuximab/trastuzumab with a range of dilutions and comparing with three controls (i. untreated cells, ii. cells treated with an isotype-matched ²¹⁴Pb/²¹⁴Bi-TCMC-control antibody that does not have EGFR1 or EGFR2 targeting but identical levels of radioactivity, iii. Cells treated with unlabeled cetuximab/trastuzumab at similar concentrations to ²¹⁴Pb/²¹⁴Bi-TCMC-trastuzumab/cetuximab) are tested.

Female dogs with bladder cancer undergo baseline clinical exam, blood CBC/clinical chemistries (liver/renal profiles), and urinalysis. UTIs are preemptively treated with antimicrobials before treatment. Inclusion criteria include that dogs must be a minimum size of 8 kg and have no urethral/ureter obstructions. The dogs receive standard of care (piroxicam) and the study treatment which consist of BCG combined with ²¹⁴Pb/²¹⁴Bi-TCMC-cetuximab/trastuzumab. The cohort includes 12 animals and each dog receives three treatments over six weeks to achieve a 20 Gy dose to the bladder wall, which is well below toxicity at 40 Gy. All treated dogs return four months after the last treatment to assess changes in tumor size (CT) and blood clinical chemistries will also be evaluated. To maximize safety, a 4-week phone follow-up with the owners are completed for every dog. If any concerns, then owners will be requested to bring dogs back before the 4-month follow-up. Dog survival will be monitored and compared with survival of dogs given standard of care only, from VMC historical records.

### Example 6: Treatment of Bladder Cancer with ²¹⁴Pb/²¹⁴Bi-Cetuximab/Trastuzumab in Humans

Patients with non-muscle invasive bladder cancer are selected and treated with either ²¹⁴Pb/²¹⁴Bi-cetuximab/trastuzumab or placebo. The therapeutic agent or placebo are administered directly to the bladder via intravesical administration. Then growth or reduction in bladder cancer is measured.

### Example 7: Delivery of ²¹⁴Pb/²¹⁴Bi to Solid Tumors with MAA

Human-approved macroaggregated (MAA) was radiolabeled with ²¹⁴Pb/²¹⁴Bi. MAA in FDA kit was mixed with ²¹⁴Pb/²¹⁴Bi in PBS. The solution was incubated by shaking for 10 minutes at 70° C. and purified by centrifugation and removal of supernatant. The ²¹⁴Pb/²¹⁴Bi-MAA was then resuspended in a human serum solution and incubated at 37° C. The purity of the product was followed with instant thin layer chromatography. The purity was greater than 97% throughout the entirety of the experiment, showing stability of the radiopharmaceutical.

The amount of energy emitted from ²¹⁴Pb/²¹⁴Bi decay was calculated using an ion chamber. ²¹⁴Pb/²¹⁴Bi-MAA at a known concentration was set on the bench top and the ion chamber placed at exactly 1 cm above the source. The ion chamber measured the mrem/hr in 3 conditions: fully open, alpha blocked, and beta blocked. This allows for approximation of mrem/hr contribution of alpha, beta, and gamma energies. It was found that approximately 75% of the energy comes from beta particles, 20% from alpha particles, and 5% from gamma particles.

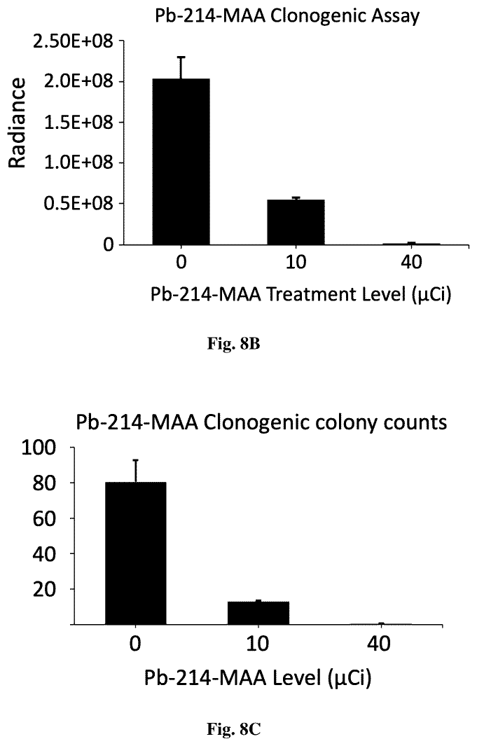

4T1 mouse breast cancer cells were plated into 6-well plates twelve hours before treatment. ²¹⁴Pb/²¹⁴Bi-MAA was added at 0 μCi (bland MAA), 10 μCi, or 40 μCi. The cells were allowed to grow for 10 days and then were imaged using luciferin based BLI and stained with crystal violet staining. Results showed that increased dose of ²¹⁴Pb/²¹⁴Bi-MAA resulted in more cell death. FIG. 8A shows the crystal violate staining of treated cells. FIG. 8B shows the total radiance from each treated sample. FIG. 8C shows the total number of colonies following manual counting of stained cells.

### Example 8: Kit and Method for Radiolabeling Trastuzumab and Cetuximab with ²¹⁴Pb and ²¹⁴Bi to Achieve Stability and Potency of the Final Drug Product (²¹⁴Pb/²¹⁴Bi-TCMC-Trastuzumab/Cetuximab)

This example demonstrated the preparation of a Kit 1 formulation for radiolabeling with ²¹⁴Pb/²¹⁴Bi harvested from a ²²²Rn manual generator. The method included rapid purification and capacity to retain strong binding of ²¹⁴Pb/²¹⁴Bi to the trastuzumab and cetuximab for 1 hour, as well as high potency of the ²¹⁴Pb/²¹⁴Bi-TCMC-Trastuzumab/Cetuximab to bind target receptors EGFR2 and EGFR1, respectively, even after 1 hour following manufacturing. Additionally, this example showed that the addition of DTPA-conjugated human serum albumin after purification of ²¹⁴Pb/²¹⁴Bi-TCMC-Trastuzumab/Cetuximab enabled scavenging (binding to DTPA-conjugated human serum albumin) of small amounts of ²¹⁴Bi that were released from the TCMC chelator during a 1-hour decay of ²¹⁴Pb (approximately 2 half-lives).

Protocol for making Kit 1: Each Kit 1 contained the following ingredients in liquid form (total volume=0.1 mL): 150 g TCMC-conjugated Trastuzumab, 150 g TCMC-conjugated Cetuximab, a solution of 35 mM ascorbate plus 35 mM gentisic acid (100 L), all contained in 0.15M ammonium acetate at pH 7.0.

TCMC-conjugated Trastuzumab-anns and TCMC-conjugated Cetuximab.

Antibodies. Trastuzumab-anns (Kanjinti) antibody is the FDA-approved biosimilar for trastuzumab (Herceptin) antibody. Cetuximab (Erbitux) is an FDA-approved chimeric antibody. The TCMC is the chelating agent for binding the ²¹⁴Pb/²¹⁴Bi. 4-NCS-Bz-TCMC is 2-(4-isothiocyanotobenzyl)-1, 4, 7, 10-tetraaza-1, 4, 7, 10-tetra-(2-carbamoyl methyl)-cyclododecane, shown below (Macrocyclics, 94% purity). The NCS portion of the molecule reacts randomly with lysine residues on the antibodies.

Conjugation. Each antibody (1-3 mg) was conjugated with 4-NCS-Bz-TCMC (hereafter TCMC) at a TCMC:antibody molar ratio of 6:1, carbonate buffer (0.1 M NaHCO₃ and 5 mM Na₂CO₃ in metal-free water) for 2 hours at 37° C. with gentle agitation. Unbound TCMC from TCMC-conjugated antibodies was removed, and carbonate buffer was exchanged by washing 3 times with 0.15M ammonium acetate using a Pierce Protein Concentrator PES (30K MWCO).

Kit 2 preparation. Each Kit 2 contained 20 mg DTPA-conjugated human serum albumin. FDA-approved human serum albumin was conjugated with p-SCN-Bn-CHX-A″-DTPA. p-SCN-Bn-CHX-A″-DTPA is [(R)-2-Amino-3-(4-isothiocyanatophenyl)propyl]-trans-(S,S)-cyclohexane-1,2-diamine-pentaacetic acid; (Macrocyclics) at a 6:1 molar ratio of DTPA:albumin for 2 hour in carbonate buffer (0.1 M NaHCO₃ and 5 mM Na₂CO₃ in metal-free water) for 2 hours at 37° C. with gentle agitation, followed by removal of unbound DTPA from DTPA-conjugated albumin and carbonate buffer exchange by washing 3 times with 0.15 M ammonium acetate using a Pierce Protein Concentrator PES (30K MWCO). A variation of Kit 2 includes gentisic acid and ascorbate for protection against radiolysis when handling greater than 5 mCi levels of ²¹⁴Pb/²¹⁴Bi.

²¹⁴Pb/²¹⁴Bi solution and radioactivity measurements. The ²¹⁴Pb/²¹⁴Bi was obtained from the manual ²²²Rn generator in 1.0 mL of 2 M ammonium acetate buffer at pH 5.3. The radioactivity levels of ²¹⁴Pb and ²¹⁴Bi were precisely measured using a calibrated Ortec High Purity Germanium (HPGe) radiation detector and a gamma-ray spectroscopy system. The HPGe detector was calibrated using a National Institute of Standards and Technology (NIST) traceable ¹⁵²Eu source in a similar geometry. ²¹⁴Pb was quantified using the 351.93 keV gamma photon, and ²¹⁴Bi was quantified using the 609.31 keV gamma photon.

Method for radiolabeling Kit 1 to produce final drug product. Example A. The ²¹⁴Pb/²¹⁴Bi solution (˜0.5-1.0 ml) was added to Kit 1, resulting in a final pH 5.3. The solution was incubated at 45° C. for 15 minutes with shaking at 750 rpm. After 15 minutes, any ²¹⁴Pb/²¹⁴Bi not bound to the antibodies were removed using a Zeba desalting spin column with the purified ²¹⁴Pb/²¹⁴Bi-TCMC-trastuzumab/cetuximab retained in phosphate-buffered saline at pH 7.0. After purification, 2 mg of DTPA-conjugated human serum albumin from Kit 2 added into the ²¹⁴Pb/²¹⁴Bi-TCMC-trastuzumab/cetuximab and was assessed as below. The results are presented in Table 3.

### Example B

In a separate experiment on a different day, the same steps were completed up to the purification step, but instead of using Kit 2, into the ²¹⁴Pb/²¹⁴Bi-TCMC-trastuzumab/cetuximab had 10 mg of human serum albumin added without any DTPA conjugated to the human serum albumin and was assessed as below. The results are presented in Table 2.

Instant Thin Layer Chromatograph (iTLC) analyses. The iTLC analyses were used to determine the ²¹⁴Pb/²¹⁴Bi tightly bound to the antibodies. After purification with the Zeb spin column, 0.001 mL of the ²¹⁴Pb/²¹⁴Bi-TCMC-trastuzumab/cetuximab was spotted at the bottom of iTLC-SG chromatography paper (glass microfiber chromatography paper impregnated with silica gel, Agilent Technologies, Cat. No. SG10001). The iTLC strip, after spotting was eluted with the following buffer: 0.15 M ammonium acetate buffer with 10 mM EDTA at pH 7.0. Unbound ²¹⁴Pb/²14Bi eluted to the top of the iTLC strip, while protein-bound ²¹⁴Pb/²¹⁴Bi remained at the bottom where it was spotted.

Immunoreactivity assay. This assay was done to determine the percentage of ²¹⁴Pb/²¹⁴Bi-TCMC-trastuzumab/cetuximab that would specifically bind to EGFR2 and EGFR1. Biotin-labeled recombinant receptors for EFGR1 (Recombinant Human EGFR Fc Chimera Avi-tag Protein, R&D Systems) and EGFR2 (Recombinant Human ErbB2/Her2 Fc Chimera Avi-tag Protein, R&D System) were attached to streptavidin-coated magnetic beads (Pierce Streptavidin Magnetic Beads, Thermo Scientific). The EGFR1 and EGFR2 coated magnetic beads were in PBS solution. The ²¹⁴Pb/²¹⁴Bi-TCMC-trastuzumab/cetuximab (˜0.2 g) was added to 2 separate tubes, one with excess added, unlabeled trastuzumab/cetuximab 30 minutes before (total each antibody=25 g), and the other without unlabeled trastuzumab/cetuximab. The solution was incubated for 5 minutes at 45° C. A magnetic tube holder (DynaMag™-2 Magnet, Thermo Scientific) was positioned to hold the beads, the liquid was removed and saved for analyses, and a separate tube with the beads was saved. The amount of ²¹⁴Pb and ²¹⁴Bi retained on the beads and in the solutions was measured.

The two variations of the binding assay, one using unlabeled, “blocking” trastuzumab/cetuximab and the other not using unlabeled trastuzumab/cetuximab, allowed calculation of the “specific” binding (immunoreactivity) of ²¹⁴Pb/²¹⁴Bi-TCMC-trastuzumab/cetuximab for EGFR1 and EGFR2 using the formula: specific binding (%) or immunoreactivity (%)=% binding of ²¹⁴Pb/²¹⁴Bi-TCMC-trastuzumab/cetuximab in the absence of unlabeled trastuzumab/cetuximab minus the % binding of ²¹⁴Pb/²¹⁴Bi-TCMC-trastuzumab/cetuximab in the presence of unlabeled trastuzumab/cetuximab. The immunoreactivity is an index of the potency of the ²¹⁴Pb/²¹⁴Bi-TCMC-trastuzumab/cetuximab.

Tables 1 and 2 present the results of various metrics for the performance of Kit 1 and Kit 2 and radiolabeling method, with (Example A) and without (Example B) DPTA-conjugated human serum albumin, respectively. The preferred method for obtaining maximum protein-bound ²¹⁴Pb/²¹⁴Bi and immunoreactivity (potency) required the addition of the DTPA-conjugated human serum albumin (Example 1 method). For both tables, note that the “²¹⁴Pb” refers to “²¹⁴Pb-TCMC-trastuzumab/cetuximab”, “¹²¹⁴Bi” refers to “¹²¹⁴Bi-TCMC-trastuzumab/cetuximab”, and “²¹⁴Pb/²¹⁴Bi” refers to, “²¹⁴Pb/²¹⁴Bi-TCMC-trastuzumab/cetuximab”.

TABLE 1
Results obtained using Kit 1 and Kit
2 for radiolabeling with ²¹⁴Pb/²¹⁴Bi

| Metric | ²¹⁴Pb | ²¹⁴Bi | ²¹⁴Pb/²¹⁴Bi |
|---|---|---|---|
| Added radioactivity (μCi) | 795 | 1,701 | 2,496 |
| Antibody Radiolabeling yield (%) | 72.90 | 30.53 | 44.03 |
| % Protein bound (iTLC) @ initial | 100.00 | 96.69 | 98.34 |
| purification (%) | | | |
| % protein bound (iTLC) @ 30 min | 100.00 | 98.71 | 99.21 |
| (%) | | | |
| % protein bound (iTLC) @ 60 min | 100.00 | 97.67 | 98.43 |
| (%) | | | |
| Immunoreactivity @ 60 min (%) | 94.77 | 74.09 | 81.01 |

TABLE 2
Results for radiolabeling with ²¹⁴Pb/²¹⁴Bi using Kit 1 with added
10 mg of human serum albumin (no Kit 2) after the purification step

| Metric | ²¹⁴Pb | ²¹⁴Bi | ²¹⁴Pb/²¹⁴Bi |
|---|---|---|---|
| Added radioactivity (μCi) | 667 | 1,479 | 2,145 |
| Antibody Radiolabeling yield (%) | 59.06 | 22.92 | 34.16 |
| % Protein bound (iTLC) @ initial | 100.00 | 86.28 | 93.74 |
| purification (%) | | | |
| % protein bound (iTLC) @ 30 min | 100.00 | 76.14 | 85.38 |
| (%) | | | |
| % protein bound (iTLC) @ 60 min | 100.00 | 73.58 | 82.49 |
| (%) | | | |
| Immunoreactivity @ 60 min (%) | 75.22 | 56.87 | 62.42 |

The kit preparation and methods achieved efficient radiolabeling of trastuzumab/cetuximab with ²¹⁴Pb/²¹⁴Bi produced stable radiolabeled antibodies that retained potency. The results presented in Table 1 show high protein binding of both ²¹⁴Pb/²¹⁴Bi after 1 hour of radiolabeling and high immunoreactivity (potency) for the binding of ²¹⁴Pb/²¹⁴Bi-TCMC-trastuzumab/cetuximab to the target EGFR2 and EGFR1, respectively.

The results presented in Table 1, compared with Table 2, also showed that DPTA-conjugated human serum albumin was necessary to keep the ²¹⁴Bi protein bound and preserve higher potency. While the potency of the ²¹⁴Pb-labeled cetuximab/trastuzumab remained high (94.77%) at 1 hour after radiolabeling/purification, the potency of the ²¹⁴Bi-cetuximab/trastuzumab was less (74.09%) because of release of part of the ²¹⁴Bi (during ²¹⁴Pb decay) which was subsequently bound by DTPA-conjugated human serum albumin. The binding of the ²¹⁴Bi was important to prevent any off-target toxicity of free ²¹⁴Bi.

### Example 9: Vacuum-Dried (Freeze-Dried) Kit System and Method for Radiolabeling Trastuzumab with ²⁰³Pb and Other Pb Radioisotopes

Making the kit system. Kit 3: The pH balance and radiolysis protection kit contained the following ingredients in liquid form (total volume=0.7 mL): 2M Ammonium acetate (pH 5.3) for pH optimization, D-mannitol (1 mg; as bulking agent) and a solution containing 35 mM ascorbate and 35 mM gentisic acid (100 microliter). Kit 4 (antibody conjugated with TCMC) contained the following ingredients: 50-100 g of TCMC-conjugated Trastuzumab in 0.15 M ammonium acetate at pH 7.0 and D-mannitol (2 mg) in liquid form. After preparation of these two kits as a liquid, the two kits vials were freeze-dried and stored at −20° C.

This example demonstrated the preparation of a two-vial kit system for pH adjustment and subsequent radiolabeling of antibody with ²⁰³Pb, ensuring radiolysis protection. In this example Kit 3 contained the buffer for pH adjustment and radiolysis protection and Kit 4 contained the TCMC-conjugated trastuzumab antibody for binding the Pb radioisotope. This example showed that a two-vial radiolabeling kit system can rapidly optimize the pH of ²⁰³Pb for subsequent binding of the ²⁰³Pb to the TCMC-conjugated trastuzumab. The kit system is also applicable for radiolabeling with ²¹²Pb or ²¹⁴Pb/²¹⁴Bi and with other antibodies and can be scaled to higher mass of antibodies to increase the amount of radioactivity that is bound.

Antibody. Trastuzumab-anns (Kanjinti, Amgen Inc.) antibody is the FDA-approved biosimilar for trastuzumab (Herceptin) antibody. Cetuximab (Erbitux, Eli Lilly and Company) is an FDA-approved chimeric antibody. The TCMC is the chelating agent for binding the ²⁰³Pb. 4-NCS-Bz-TCMC is 2-(4-isothiocyanotobenzyl)-1, 4, 7, 10-tetraaza-1, 4, 7, 10-tetra-(2-carbamoyl methyl)-cyclododecane (Macrocyclics; 94% purity). The NCS portion of the molecule randomly reacts with lysine residues on the antibodies.

Conjugation. Each antibody (1-3 mg) was conjugated with 4-NCS-Bz-TCMC (hereafter TCMC) at a TCMC: Antibody molar ratio of 6:1, in carbonate buffer (0.1 M NaHCO₃ and 5 mM Na2CO3 in metal-free water, pH=8.5-9.0) for 2 hours at 37° C. with gentle agitation. Unbound TCMC and carbonate buffer was removed from TCMC-conjugated antibody by washing 3 times with 0.15M ammonium acetate (pH=7.0) using a Pierce Protein Concentrator PES (30K MWCO).

²⁰³Pb solution and radioactivity measurements. The ²⁰³Pb was purchased from the University of Alabama, Birmingham. The radioactivity levels of ²⁰³Pb were measured using a calibrated dose calibrator (CRC-25R, Capintec). The dose calibrator was calibrated using a National Institute of Standards and Technology (NIST) traceable Cs-137 source in a similar geometry. ²⁰³Pb was quantified using the 838.5 keV gamma photon.

Method and variations tested for radiolabeling using two-vial kit system to produce ²⁰³Pb-TCMC-trastuzumab. Example A. Evaluation of the two-kit system with different microcuries levels of ²⁰³Pb relative to micrograms levels of TCMC-conjugated trastuzumab, and different temperatures. In two separate experiments ²⁰³Pb solution (˜0.05-0.1 ml; 500 or 1000 μCi) was added to Kit 3, resulting in a solution of final pH at 5.3. Next, the entire reaction solution from Kit 3 was added to Kit 4 (antibody conjugated with TCMC) and mixed.

In one experiment, a total of 500 μCi of ²⁰³Pb was added to Kit 4 that contained 100 g of TCMC-conjugated Trastuzumab, to provide a ratio of 5:1. In a separate experiment, a total of 1000 μCi of ²⁰³Pb was added to Kit 4 that contained 100 g of TCMC-conjugated Trastuzumab, to provide a ratio of 10:1. In each of the two examples with 500 or 1000 μCi the solution was incubated at 45° C. with shaking at 750 rpm for 15 minutes and tested by iTLC, and then tested again by iTLC after 30 minutes after incubation. The steps were repeated in separate experiments with separate kits, with incubation at 37° C., 55° C., and 60° C. The ²⁰³Pb-TCMC-trastuzumab was not further purified, rather it was tested for immunoreactivity (binding assay) as described below.

### Example B. Evaluation of Two-Kit System for Conjugation of ²⁰³Pb to TCMC-Conjugated

trastuzumab at 45° C. ²⁰³Pb solution (˜0.05-0.1 ml; 500 μCi) was added to Kit 3, resulting in a solution of final pH at 5.3. Next, the entire reaction solution from Kit 3 was added to Kit 4 (100 microgram Trastuzumab conjugated with TCMC) and mixed. The solution was incubated at 45° C. with shaking at 750 rpm for 30 minutes and tested by iTLC. The ²⁰³Pb-TCMC-trastuzumab was not further purified, rather it was tested for stability in human serum at 37° C. over a 22-hour period by iTLC.

Instant Thin Layer Chromatograph (iTLC) analyses determined the binding of ²⁰³Pb to the TCMC-trastuzumab. The iTLC analyses were used to determine the ²⁰³Pb tightly bound to the antibodies. After radiolabeling, 0.001 mL of the ²⁰³Pb-TCMC-trastuzumab was spotted at the bottom of iTLC-SG chromatography paper (glass microfiber chromatography paper impregnated with silica gel, Agilent Technologies, Cat. No. SG10001). The iTLC strip, after spotting, was eluted with the following buffer: 0.15 M ammonium acetate buffer with 10 mM EDTA at pH 7.0. Unbound ²⁰³Pb eluted to the top of the iTLC strip, while protein-bound ²⁰³Pb remained at the bottom where it was originally spotted.

Serum stability assay. This assay was done to determine the percentage of ²⁰³Pb that remained bound to TCMC-trastuzumab, when incubated in human serum. After radiolabeling at 45° C., 10 μL of ²⁰³Pb-TCMC-trastuzumab was added into 90 μL of human serum and incubated at 37° C. with gentle shaking. iTLC was performed at 0-, 1-, 2-, 3-, 4-, 5-, 6- and 22-hour timepoints with incubation at 37° C. during the 0-22 hr period.

Binding assay (always conducted at 37° C.) to determine antibody binding capacity (immunoreactivity) after radiolabeling trastuzumab at 37° C. (30 minutes), 45° C. (15 and 30 minutes), 55° C. (5 minutes), and 60° C. (5 minutes). For binding assay, HER2+ human ovarian cancer cells (SKOV3) were seeded in a 96-well plate (30,000 cells/well) at 37° C. and 5% CO₂ overnight. Cells were washed with PBS and ²⁰³Pb-TCMC-trastuzumab was added at 6 concentrations (30, 15, 7.5, 3.75, 1.88, and 0.94 nM) for radiolabeling at 37° C., 45° C., 55° C. and 60° C. ²⁰³Pb-TCMC-trastuzumab was added at 8 concentrations (30, 15, 7.5, 3.75, 1.88, 0.94, 0.47, and 0.23 nM), either alone (unblocked condition) or with 100-fold excess unlabeled trastuzumab (blocked condition) and cells were incubated at 37° C. for 1 hour with the ²⁰³Pb-TCMC-trastuzumab. Next, cells were washed with PBS three times, lysed with 1M NaOH (0.1 mL). Further, the bound ²⁰³Pb-TCMC-trastuzumab to the cells was measured in a gamma counter. The binding affinity (equilibrium dissociation constant, Kd) were calculated by nonlinear regression curve analysis using GraphPad Prism (GraphPad by Dotmatics, Boston, MA).

Evaluation of the two-kit system with different μCi levels of ²⁰³Pb relative to micrograms levels of TCMC-conjugated trastuzumab. Radiolabeling was performed at two ratios: 5:1 and 10:1 for ²⁰³Pb activity (μCi) relative to micrograms of TCMC-trastuzumab amount. For 5:1 ratio the iTLC results showed 99.55% of ₂₀₃Pb was bound to TCMC-trastuzumab at 15 minutes of incubation at 45° C., and 99.98% of the ²⁰³Pb was bound to TCMC-trastuzumab at 30 minutes of incubation at 45° C. For 10:1 ratio the iTLC results showed 98.04% of ²⁰³Pb was bound to TCMC-trastuzumab at 15 minutes of incubation at 45° C., and 99.68% of the ²⁰³Pb was bound to TCMC-trastuzumab at 30 minutes of incubation at 45° C. For ²⁰³Pb radiolabeling with TCMC conjugated trastuzumab at 37° C., 55° C., and 60° C., the iTLC results showed 98.81% of ²⁰³Pb was bound to TCMC-trastuzumab after 30 minutes of incubation at 37° C., 99.82% of the ²¹³Pb was bound to TCMC-trastuzumab after 5 minutes of incubation at 55° C., and 99.85% of the ²⁰³Pb was bound to TCMC-trastuzumab after 5 minutes of incubation at 60° C.

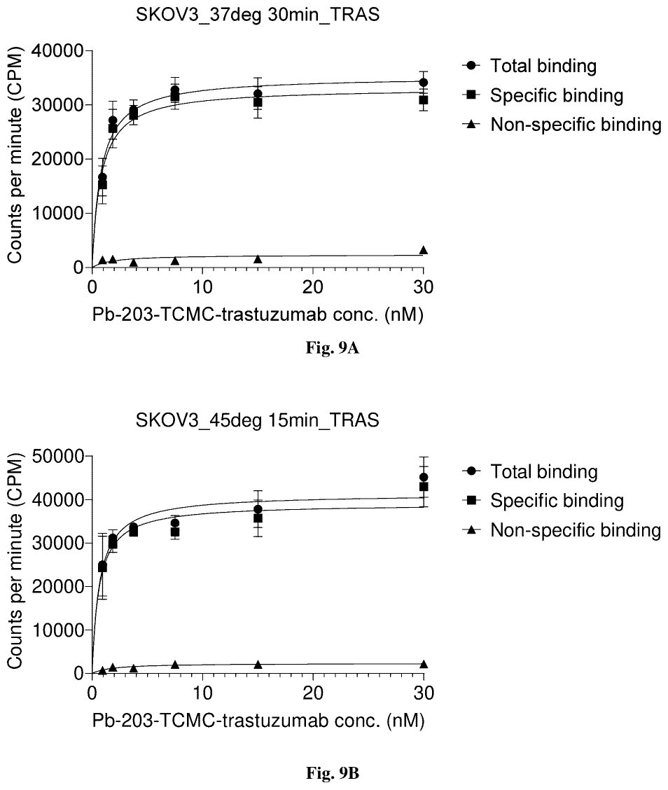

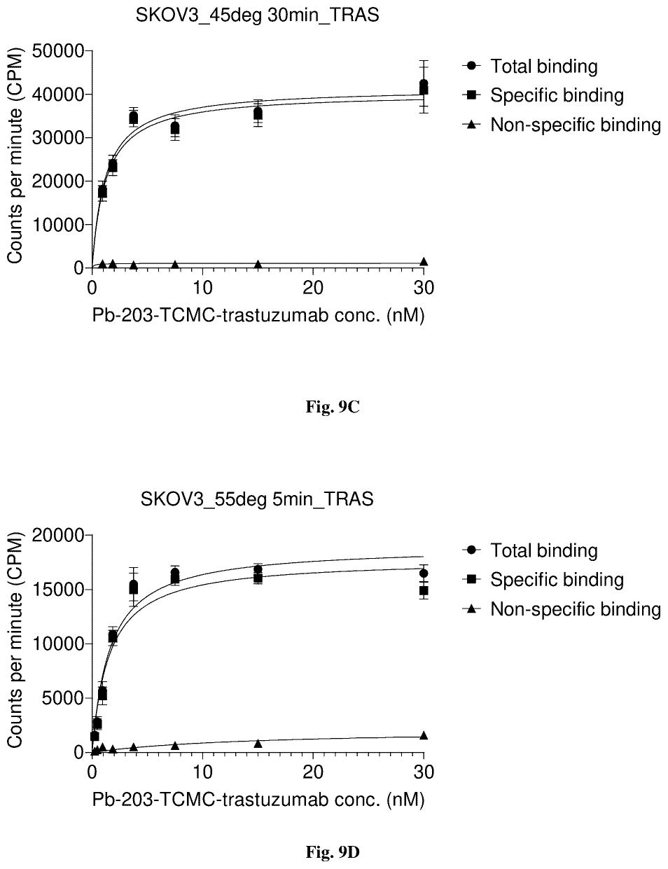

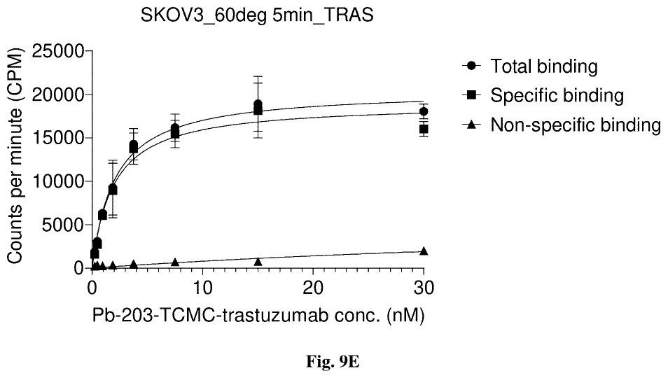

FIG. 9A-E presents the saturation binding curves for the binding of ²⁰³Pb-TCMC-trastuzumab to the HER2+SKOV3 cells (human ovarian cancer cell line) after radiolabeling at different temperatures and durations (370 for 30 minutes; 45° C. for 15 minutes; 45° C. for 30 minutes; 55° C. for 5 minutes; and 60° C. for 5 minutes. The results indicated that the ²⁰³Pb-TCMC-trastuzumab retained high specific binding to the SKOV3 cells (high immunoreactivity) for different temperature range used for radiolabeling. Kd values (equilibrium dissociation constant) calculated from the binding assays presented in FIG. 9A-E were 2 nM or less. The non-specific binding assay had unlabeled blocking levels of trastuzumab and further validated the high immunoreactivity of the ²⁰³Pb-TCMC-trastuzumab.

Evaluation of two-kit system for conjugation of ²⁰³Pb to TCMC-conjugated trastuzumab at 37° C. The serum stability assay was used to determine the percentage of ²⁰³Pb that remained bound to TCMC-trastuzumab when incubated in human serum. The percentage of ²⁰³Pb bound to TCMC-trastuzumab was 99.3%, 99.11%, 99.00%, 98.75%, 99.01%, 99.14%, 99.43%, and 99.18% at 0, 1 hr, 2 hr, 3 hr, 4 hr, 5 hr, 6 hr, and 22 hr, respectively.

### Example 10: Kit and Method for Radiolabeling Cetuximab with ²¹²Bi

This EXAMPLE demonstrates the preparation of a radiolabeling kit formulation of cetuximab for radiolabeling with ²¹²Bi. The method includes radiolabeling of ²¹²Bi with a DTPA-cetuximab conjugate in a special formulation with pH optimization and radiolysis protection. This kit and method are also applicable for radiolabeling other Bismuth radioisotopes, such as ²¹³Bi and ²¹⁴Bi.

Radiolabeling kit preparation. The pH balance kit (Vial 1) contained the following ingredients in liquid form (total volume=0.4 mL): 1M Ammonium acetate (pH 5.5) for pH balance and 35 mM ascorbate and gentisic acid (radio protectant). The antibody-chelator conjugate kit (vial 2) contained 50-100 micrograms of DTPA-conjugated cetuximab in 0.15M ammonium acetate at pH 7.0 in liquid form.

Antibodies. Cetuximab (Erbitux) is an FDA-approved chimeric antibody and was obtained from the Michigan State University pharmacy. The p-SCN-Bn-CHX-A″-DTPA is the chelating agent for binding the ²¹²Bi. p-SCN-Bn-CHX-A″-DTPA is [(R)-2-Amino-3-(4-isothiocyanatophenyl)propyl]-trans-(S,S)-cyclohexane-1,2-diamine-pentaacetic acid (Macrocyclics at 94% purity). The NCS portion of the molecule randomly reacts with lysine residues on the antibodies.

Conjugation. Each antibody (1-3 mg) was conjugated with p-SCN-Bn-CHX-A″-DTPA (hereafter DTPA) at a DTPA: Antibody molar ratio of 5, carbonate buffer (0.1 M NaHCO₃ and 5 mM Na₂CO₃ in metal-free water) for 2 hours at 37° C. with gentle agitation. Unbound DTPA from DTPA-conjugated antibodies was removed, and carbonate buffer was exchanged by washing 3 times with 0.15M ammonium acetate using a Pierce Protein Concentrator PES (30K MWCO).

²¹²Bi solution and radioactivity measurements. ²¹²Bi was obtained from the ²²⁴Ra/²¹²Pb generator provided by the Pacific Northwest National Laboratory (PNNL). ²¹²Bi was eluted from ²²⁴Ra/²¹²Pb generator using 2M HCl and trapping the ²¹²Pb in Pb-resin column preconditioned with 2M HCl (100 mg of 25-50 um (f-grade) lead resin (Eichrom Technologies Inc.) and flow through from Pb-resin column had pure ²¹²Bi. The radioactivity levels of ²¹²Bi were measured using a calibrated dose calibrator (CRC-25R, Capintec). The dose calibrator was calibrated using a National Institute of Standards and Technology (NIST) traceable Cs-137 source in a similar geometry. ²¹²Bi was quantified using the 727.3 keV gamma photon.

Method for radiolabeling using two vial kits to produce the final product. The ²¹²Bi solution (˜0.05-0.1 ml; 500-1000 microcurie) was added to the pH balance kit (vial 1), resulting in a final pH 5.5. Next, the whole reaction mix from vial 1 was added to vial 2 (Antibody-chelator conjugate kit), and The solution was incubated at 37° C. for 30-60 minutes with shaking at 750 rpm. After 60 minutes, any ²¹²Bi not bound to the antibodies were removed using a Zeba desalting spin column with the purified ²¹²Bi-DTPA-cetuximab retained in phosphate-buffered saline at pH 7.0.

Instant Thin Layer Chromatograph (iTLC) analyses to determine the binding of ²¹²Bi to the DTPA-cetuximab. The iTLC analyses were used to determine the ²¹²Bi tightly bound to the antibodies. After radiolabeling, 0.001 mL of the ²¹²Bi-DTPA-cetuximab was spotted at the bottom of iTLC-SG chromatography paper (glass microfiber chromatography paper impregnated with silica gel, Agilent Technologies, Cat. No. SG10001). The iTLC strip, after spotting was eluted with the following buffer: 0.1M sodium citrate. Unbound ²¹²Bi eluted to the top of the iTLC strip, while antibody-bound ²¹²Bi remained at the bottom, where it was spotted.

The two-vial radiolabeling kit for radiolabeling ²¹²Bi maintained the proper pH 5.5 for radiolabeling the DTPA-cetuximab conjugate. Prior to Zeba spin column purification, the iTLC results showed that for 30 minutes of incubation at 37° C., 44.01% of added ²¹²Bi were tightly bound to the DTPA-cetuximab conjugate. After 60 minutes of incubation at 37° C., 69.03% of added ²¹²Bi were tightly bound to the DTPA-cetuximab conjugate. After Zeba desalting spin column purification and at 60 minutes after radiolabeling, the percentage of ²¹²Bi tightly bound to the DTPA-cetuximab was 98.44%.

### Example 11: Kit and Radiolabeling Method to Bind ²¹²Bi, ²¹⁴Bi, or ²¹⁴Pb/²¹⁴Bi to Macro Aggregated Albumin (MAA)

These examples demonstrated the preparation of a radiolabeling kit for binding ²¹²Bi or ²¹⁴Bi to MAA. The method includes rapid and stable binding of ²¹²Bi, ²¹⁴Bi, or ²¹⁴Pb/²¹⁴Bi to MAA. This example showed that a radiolabeling kit can enable rapid radiolabeling of MAA with an alpha-emitting radioisotope. The radiolabeled MAA can be used as a vehicle for alpha-particle delivery to the tumor by interventional radiology procedures.

Kit 5. The MAA radiolabeling kit 1 contained the following ingredients in liquid form (total volume=0.4 mL): 0.2M Sodium acetate (pH 4.3) for pH balance and 0.5 milligrams MAA in liquid form. Kit 6. The MAA radiolabeling kit 2 contained the following ingredients in liquid form (total volume=0.4 mL): 10M sodium hydroxide and 4M ammonium acetate (pH 5.5) for pH balance, 35 mM ascorbate and gentisic acid for radioprotection and DTPA-conjugated MAA in 1× phosphate buffer saline at pH 7.0 in liquid form.

DTPA-conjugated MAA preparation. Macro Aggregated Albumin (Pulmotech) is an FDA-approved imaging agent that is with Tc-99m. The p-SCN-Bn-CHX-A″-DTPA is the chelating agent for binding the ²¹⁴Bi. p-SCN-Bn-CHX-A″-DTPA is [(R)-2-Amino-3-(4-isothiocyanatophenyl)propyl]-trans-(S,S)-cyclohexane-1,2-diamine-pentaacetic acid (Macrocyclics at 94% purity). The NCS portion of the molecule reacted randomly with lysine residues on the MAA.

Conjugation. MAA (0.5 milligram) was conjugated with p-SCN-Bn-CHX-A″-DTPA (hereafter DTPA) at a DTPA: MAA molar ratio of 6:1, carbonate buffer (0.1 M NaHCO₃ and 5 mM Na₂CO₃ in metal-free water) for 1 hour at 37° C. with agitation (1000 rpm). Unbound DTPA from DTPA-conjugated antibodies was removed, and carbonate buffer was exchanged by washing three times with phosphate buffer saline and centrifugation at 5000 rpm for 5 minutes.

²¹²Bi and ²¹⁴Bi solutions and radioactivity measurements. ²¹²Bi was obtained from the ²²⁴Ra/²¹²Pb generator provided by the Pacific Northwest National Laboratory (PNNL). ²¹²Bi was eluted from ²²⁴Ra/²¹²Pb generator using 2M HCl and trapping the ²¹²Pb in Pb-resin column preconditioned with 2M HCl (100 mg of 25-50 um (f-grade) lead resin (Eichrom Technologies Inc.) and flow through from the Pb-resin column was pure ²¹²Bi. The radioactivity levels of ²¹²Bi were measured using a calibrated dose calibrator (CRC-25R, Capintec). The dose calibrator was calibrated using a National Institute of Standards and Technology (NIST) traceable Cs-137 source in a similar geometry. ²¹²Bi was quantified using the 727.3 keV gamma photon. ²¹⁴Bi was eluted from the manual ²²²Rn generator. First, ²¹⁴Pb and ²¹⁴Bi were collected in 1 milliliter of 0.1M HCl from the ²²²Rn generator and passed through a Pb-resin column preconditioned with 2M HCl (100 mg of 25-50 um (f-grade) lead resin; Eichrom Technologies Inc.) to trap ²¹⁴Pb and flow through from the Pb resin column was pure ²¹⁴Bi. ²¹⁴Bi was precisely measured using a calibrated Ortec High Purity Germanium (HPGe) radiation detector and a gamma-ray spectroscopy system. ²¹⁴Bi was quantified using the 609.31 keV gamma photon.

### Example A

Method using the two-vial kit system to produce ²¹²Bi-MAA. The ²¹²Bi solution (˜0.05-0.1 ml; 500-1000 microcurie) was added to the pH balance kit 7 (10M sodium hydroxide and 0.2M sodium acetate pH 4.3), resulting in a final pH of 4.3. Next, 500 micrograms of MAA were added from purchased Macro Aggregated Albumin (Pulmotech), and the mixture was incubated at 70° C. for 10 minutes with shaking at 1000 rpm. After radiolabeling, any ²¹²Bi not bound to the MAA was removed by centrifugation at 5000 rpm and washing 3 times with phosphate buffer saline pH 7.0.

### Example B

Method to radiolabel MAA with ²¹⁴Pb/²¹⁴Bi of Example 4 above and results are shown in FIGS. 8A, 8B, 8C.

### Example C

Method to produce ²¹⁴Bi-DTPA-MAA. A ²¹⁴Bi solution (0.5 mL; 500-1000 microcurie) was added to a pH balance solution (vial 1; 10M sodium hydroxide and 4M ammonium acetate, pH 5.5). After pH balance, kit 8 containing DTPA-MAA conjugate was added and incubated at 45° C. for 15 minutes and 750 rpm. To remove unlabeled ²¹⁴Bi, the reaction mixture was centrifuged at 2000 rpm for 2 minutes, and the supernatant was removed. Next, the pellet containing ²¹⁴Bi-DTPA-MAA was resuspended in phosphate buffer saline and radiolabeling purity (% of ²¹⁴Bi tightly bound to MAA) was checked by iTLC.

Instant Thin Layer Chromatograph (iTLC) analyses to determine the binding of ²¹²Bi to the MAA and ²¹⁴Pb to the DTPA-MAA. The iTLC analyses were used to determine the ²¹²Bi and ²¹⁴Bi tightly bound to the MAA. After radiolabeling, 0.001 mL of the ²¹²Bi-MAA and ²¹⁴Bi-DTPA-MAA were spotted at the bottom of iTLC-SG chromatography paper (glass microfiber chromatography paper impregnated with silica gel, Agilent Technologies, Cat. No.

SG10001). The iTLC strip, after spotting at the bottom, was eluted with the following buffer: 0.1M sodium citrate (pH=7.6). Unbound ²¹²Bi and ²¹⁴Bi eluted to the top of the iTLC strip, while MAA and DTPA-MAA-bound ²¹²Bi and ²¹⁴Bi remained at the bottom, where it was spotted.

Example A: The method resulted in 58.49% radiolabeling yield for attachment of ²¹²Bi to MAA, and purity by iTLC was 99.13%.

Example B: See Example 4 above and FIGS. 8A, 8B, and 8C.

Example C: The kits and method resulted in 64% radiolabeling yield for ²¹⁴Bi attachment, and the radiolabeling purity by iTLC showed 100% purity.

### Example 12

One-vial vacuum-dried (freeze-dried) kit system and method for radiolabeling trastuzumab with ²¹²Pb and ²⁰³Pb radioisotopes. This example demonstrated the preparation of a one-vial kit system for pH adjustment and subsequent radiolabeling of antibody with ²¹²Pb and ²⁰³Pb with radiolysis protection. In this example, one vial kit contained the buffer for pH adjustment and radiolysis protection, bulking agent, cryoprotectant/lyoprotectant, surfactant to protect antibody from aggregation and denaturation, and TCMC-conjugated trastuzumab antibody for binding the Pb radioisotope. This example showed that a one-vial radiolabeling kit system can rapidly optimize the pH of ²⁰³Pb and ²¹²Pb and subsequent binding to TCMC-conjugated trastuzumab. The kit system is also applicable for radiolabeling with ²¹⁴Pb/²¹⁴Bi and with other antibodies.

Making the kit system. One vial kit: This kit contained the following ingredients in liquid form (total volume=0.7 mL): 2M Ammonium acetate (pH 5.3) for pH optimization, D-mannitol (3 millgrams; as a bulking agent) and a solution containing 35 mM ascorbate and 35 mM gentisic acid (50 μL; radiolysis protectant), D-mannitol (9.15 milligrams; bulking agent), 8.5 mg sucrose (cryoprotectant/lyoprotectant), 50 micrograms of polysorbate 80 (protect antibody from aggregation and denaturation) and trastuzumab conjugated with TCMC (50-100 micrograms of TCMC-conjugated Trastuzumab in 0.15M ammonium acetate at pH 7.0 in liquid form. After the preparation of this solution in a single vial kit as a liquid, vial was freeze-dried and stored at −20° C.

Antibody. Trastuzumab-anns (Kanjinti, Amgen Inc.) antibody is the FDA-approved biosimilar for trastuzumab (Herceptin) antibody. The TCMC is the chelating agent for binding the ²⁰³Pb and ²¹²Pb. 4-NCS-Bz-TCMC is 2-(4-isothiocyanotobenzyl)-1, 4, 7, 10-tetraaza-1, 4, 7, 10-tetra-(2-carbamoyl methyl)-cyclododecane (Macrocyclics at 94% purity). The NCS portion of the molecule reacted randomly with lysine residues on the antibodies.

Conjugation. Trastuzumab (1-3 mg) was conjugated with 4-NCS-Bz-TCMC (hereafter TCMC) at a TCMC: Antibody molar ratio of 6:1, in carbonate buffer (0.1 M NaHCO₃ and 5 mM Na₂CO₃ in metal-free water, pH=8.5-9.0) for 2 hours at 37° C. with gentle agitation. Unbound TCMC and carbonate buffer was removed from TCMC-conjugated antibody by washing 3 times with 0.15M ammonium acetate (pH=7.0) using a Pierce Protein Concentrator PES (30K MWCO).

²¹²Pb and ²⁰³Pb solution and radioactivity measurements. The ²²⁴Ra/²¹²Pb generator was provided by the Pacific Northwest National Laboratory (PNNL). ²¹²Pb was eluted from ²²⁴Ra/²¹²Pb generator using 2M HCl and trapping the ²¹²Pb in Pb-resin column preconditioned with 2M HCl (100 mg of 25-50 um (f-grade) lead resin; Eichrom Technologies Inc.). Next, trapped ²¹²Pb was eluted from the Pb-resin column using 1M sodium acetate pH 5.3. The radioactivity levels of ²¹²Pb were measured using a calibrated dose calibrator (CRC-25R, Capintec). The dose calibrator was calibrated using a National Institute of Standards and Technology (NIST) traceable Cs-137 source in a similar geometry. ²¹²Pb was quantified using the 238.6 keV gamma photon. The ²⁰³Pb was purchased from the University of Alabama, Birmingham. The radioactivity levels of ²⁰³Pb were measured using a calibrated dose calibrator (CRC-25R, Capintec). The dose calibrator was calibrated using a National Institute of Standards and Technology (NIST) traceable Cs-137 source in a similar geometry. ²⁰³Pb was quantified using the 838.5 keV gamma photon.

### Example a. Evaluation of the One-Vial Radiolabeling Kit System for ²¹²Pb Radiolabeling with TCMC-Conjugated Trastuzumab

²¹²Pb solution (˜0.05-0.1 ml; 550 μCi) was added to one vial radiolabeling kit containing 50 micrograms of TCMC-trastuzumab. Next, the reaction solution was incubated at 45° C. with shaking at 750 rpm for 15 minutes and tested by iTLC, and then tested again by iTLC after 30 minutes after incubation. The ²¹²Pb-TCMC-trastuzumab was further purified Zeba desalting spin column with the purified ²¹²Pb-TCMC-trastuzumab/cetuximab retained in phosphate buffered saline at pH 7.0. Radiolabeling purity was further tested by iTLC.

### Example B. Evaluation of the One-Vial Radiolabeling Kit System for ²⁰³Pb Radiolabeling with TCMC-Conjugated Trastuzumab after Five Months of Kit Preparation and Storage at −20° C.

After one-vial radiolabeling kit preparation by vacuum-dried (freeze-dried) method, kits were stored at −20° C. A solution of 250 μCi of ²⁰³Pb was added to a five-month-old one-vial radiolabeling kit, mixed, and heated at 45° C. for 15 minutes at gentle agitation. Radiolabeling purity was further tested by iTLC.

Assessment of Stability and Potency of the final radiolabeled product (²⁰³Pb-TCMC-trastuzumab). Instant Thin Layer Chromatograph (iTLC) analyses determined the binding of ²¹²Pb/²⁰³Pb to the TCMC-trastuzumab. The iTLC analyses were used to determine the ²¹²Pb tightly bound to the antibodies. After radiolabeling, 0.001 mL of the ²¹²Pb/²⁰³Pb-TCMC-trastuzumab was spotted at the bottom of iTLC-SG chromatography paper (glass microfiber chromatography paper impregnated with silica gel, Agilent Technologies, Cat. No. SG10001). The iTLC strip, after spotting was eluted with the following buffer: 0.15 M ammonium acetate buffer with 10 mM EDTA at pH 7.0. Unbound ²¹²Pb/²⁰³Pb eluted to the top of the iTLC strip, while trastuzumab-bound ²¹²Pb/²⁰³Pb remained at the bottom where it was spotted.

Serum stability assay. This assay was done to determine the percentage of ²⁰³Pb that remained bout to TCMC-trastuzumab, when incubated in human serum. After radiolabeling, L of ²⁰³Pb-TCMC-trastuzumab were added into 90 μL of human serum and incubated at 37° C. with gentle shaking. iTLC was performed at 0-, 1-, 2-, 3-, 4-, 5- and 24-hour timepoints.

### Example a: Evaluation of the One Vial-Kit System for ²¹²Pb Radiolabeling with TCMC-Conjugated Trastuzumab

Radiolabeling was performed by adding 550 μCi of ²¹²Pb in one vial radiolabeling kit having all components needed for radiolabeling, including 50 micrograms of TCMC conjugated trastuzumab. After that reaction, the solution was heated at 45° C., and iTLC analyses showed 42.93% and 54.14% radiolabeling yield at 15 and 30 minutes of incubation, respectively. After Zeba column purification, 97.71% radiolabeling purity was achieved for the ²¹²Pb-TCMC-trastuzumab.

### Example B

Evaluation of the one-vial radiolabeling kit system for ²⁰³Pb radiolabeling with TCMC-conjugated trastuzumab after five months of kit preparation and storage at −20° C.: After 15 minutes of radiolabeling at 45° C., iTLC showed 99.84% radiolabeling of ²⁰³Pb with TCMC conjugated trastuzumab. Further, the serum stability assay showed 99.84%, 99.79%, 99.84%, 99.68%, 99.59%, and 99.64% of ²⁰³Pb remained tightly bound as ²⁰³Pb-TCMC-trastuzumab after 1-, 2-, 3-, 4-, 5- and 24-hours of radiolabeling, respectively.

The use of the terms “a” and “an” and “the” and “at least one” and similar referents in the context of describing the fusion peptide and related uses (especially in the context of the following claims) are to be construed to cover both the singular and the plural, unless otherwise indicated herein or clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Particular embodiments of the conjugate are described herein, including the best mode known to the inventors for carrying out the invention. Variations of those particular embodiments may become apparent to those of ordinary skill in the art upon reading the foregoing description. The inventors expect skilled artisans to employ such variations as appropriate, and the inventors intend for the conjugate to be practiced otherwise than as specifically described herein. Accordingly, the conjugate described herein includes all modifications and equivalents of the subject matter recited in the claims appended hereto as permitted by applicable law. Moreover, any combination of the above-described elements in all possible variations thereof is encompassed by the described fusion peptide unless otherwise indicated herein or otherwise clearly contradicted by context.

What is claimed is:

1. A conjugate comprising an anti-cancer agent and an alpha-particle emitter, wherein the alpha-particle is selected from the group consisting of ²¹⁴Pb, ²¹²Bi, ²¹⁴Bi, ²¹⁴Pb/²¹²Pb, ²¹⁴Pb/²¹⁴Bi, and ²¹²Pb/²¹²Bi.

2. The conjugate of claim 1, wherein the alpha-particle emitter is ²¹⁴Pb/²¹⁴Bi.

3. The conjugate of claim 1, wherein the anti-cancer agent comprises a HER2 inhibitor, an EGFR inhibitor, macroaggregated albumin (MAA), or a combination thereof.

4. The conjugate of claim 3, wherein the HER2 inhibitor comprises trastuzumab, trastuzumab-anns, trastuzumab-dkst, trastuzumab-qyyp, or trastuzumab-pkrb.

5. The conjugate of claim 4, wherein the HER2 inhibitor comprises trastuzumab.

6. The conjugate of claim 3, wherein the EGFR inhibitor comprises cetuximab, panitumumab, zalutumumab, nimotuzumab, or matuzumab.

7. The conjugate of claim 1, further comprising a chelator.

8. The conjugate of claim 7, wherein the chelator comprises 2-[4,7,10-tris(2-amino-2-oxoethyl)-1,4,7,10-tetrazacyclododec-1-yl]acetamide (TCMC), 2,2′,2″,2′″-(1,4,7,10-Tetraazacyclododecane-1,4,7,10-tetrayl)tetraacetic acid (DOTA), or 2,2′,2″,2′″-{[(Carboxymethyl)azanediyl]bis(ethane-2,1-diylnitrilo)}tetraacetic acid (DTPA).

9. The conjugate of claim 7, wherein the anti-cancer agent comprises trastuzumab, the alpha-particle emitter comprises ²¹⁴Pb/²¹⁴Bi, and the chelator comprises TCMC.

10. The conjugate of claim 1, wherein the anti-cancer agent comprises MAA and the alpha-particle emitter comprises ²¹⁴Pb/²¹⁴Bi.

11. The conjugate of claim 1, wherein the anti-cancer agent comprises cetuximab and trastuzumab, and the alpha-particle emitter comprises ²¹⁴Pb/²¹⁴Bi.

12. A pharmaceutical composition comprising the conjugate of claim 1 and one or more pharmaceutically acceptable excipients.

13. The pharmaceutical composition of claim 12, wherein the one or more pharmaceutically acceptable excipients comprise a pH buffer and a stabilizer.

14. The pharmaceutical composition of claim 12, wherein the anti-cancer agent comprises trastuzumab.

15. The pharmaceutical composition of claim 14, wherein the alpha-particle emitter comprises ²¹⁴Pb/²¹⁴Bi, and the conjugate further comprises a chelator comprising TCMC or DOTA.

16. A kit comprising an alpha-particle emitter and an anti-cancer agent.

17. The kit of claim 16, wherein the kit comprises a first vial and a second vial, wherein:
the first vial comprises the alpha-particle emitter; and
the second vial comprises the anti-cancer agent.

18. The kit of claim 17, wherein the first vial and the second vial independently further comprise a buffer for pH adjustment and radiolysis protection, a bulking agent, a cryoprotectant/lyoprotectant, a surfactant, or a combination thereof.

19. The kit of claim 16, wherein the kit comprises a single vial.

20. The kit of claim 19, wherein the single vial further comprises a buffer for pH adjustment and radiolysis protection, a bulking agent, a cryoprotectant/lyoprotectant, a surfactant, or a combination thereof.

21. The kit of claim 16, wherein the kit comprises shielding material to block or inhibit radiation from the alpha-particle emitter.
